(19) United States
(12) Patent Application Publication
(10) Pub. No.: US 2026/0144665 A1
(43) Pub. Date: May 28, 2026

(54) Customizable Inflatable Bolster Apparatus

(71) Applicant: Vanderbilt University, Nashville, TN (US)

(72) Inventors: Alexander Jay Langerman, Nashville, TN (US); Kavita Prasad, Nashville, TN (US)

(21) Appl. No.: 19/121,984

(22) PCT Filed: Oct. 23, 2023
(86) PCT No.: PCT/US2023/077518

§ 371 Date: Apr. 17, 2025

Related U.S. Application Data
(60) Provisional application No. 63/418,389, filed on Oct. 21, 2022.

(51) Int. Cl. A61F 5/34; A61F 5/32; A61K 35/36; A61M 35/00

(52) U.S. Cl. CPC A61F 5/34; A61F 5/32; A61K 35/36; A61M 35/10

(57) ABSTRACT

A customizable inflatable bolster apparatus (100) may include a first leaf (102), a second leaf (104), or a balloon (108) disposed between the first leaf (102) and the second leaf (104) or on a single leaf. The balloon (108) may include an inflation port (110) and one or more tethers (112)(1)-(n)). The one or more tethers (112)(1)-(n)) may be disposed within the balloon (108) or around an external surface of the balloon (108). In response to the balloon (108) inflating, the one or more tethers ((112)(1)-(n)) may shape the balloon (108) into a desired shape. The apparatus (100) may apply a pressure to the tissue of patient and immobilize the tissue. The apparatus (100) may be applied to a skin graft, a patient's tissue, or a mucosa or fascia of the patient. The apparatus (100) may elute a substance, such as a pharmaceutical, into the tissue of the patient.

### CROSS-REFERENCE TO RELATED APPLICATIONS

This application is a non-provisional application and claims priority to U.S. Provisional Patent Application No. 63/418,389, filed Oct. 21, 2022, entitled CUSTOMIZABLE INFLATABLE BOLSTER DEVICE, which is hereby incorporated by reference in its entirety.

### FEDERALLY SPONSORED RESEARCH OR DEVELOPMENT

Not applicable.

### BACKGROUND

The present disclosure The present disclosure generally relates to medical supplies, and more particularly to a customizable inflatable bolster apparatus.

There are many medical scenarios for applying a medical supply to a patient's tissue. One scenario may include applying pressure to a designated area of the human body in order to immobilize tissue and obliterate voids. Such scenarios include a skin graft, maintaining a material against the skin or mucosa, compressing a wound cavity, and preventing bleeding from a wound. In another scenario, a medical supply may be adhered to a patient's tissue so a substance disposed within the medical supply can elute into the patient's tissue, or alternatively, the medical supply can draw away substances from the wound bed.

Conventional means for applying such pressure to the tissue is a rolled-up piece of gauze placed over the area and applying a bandage over the gauze, tying sutures over the gauze, or suturing the edges of the gauze to keep the gauze in place. This conventional means has several disadvantages. First, there is very little control over the amount of pressure applied to the tissue, particularly if the tissue bed is irregular. Second, the gauze is absorbent and can quickly harbor bacteria, leading to infection, inflammation, or unpleasant odor. It can also dry out the graft, which can lead the gauze to adhere to the graft and dislodge it when the gauze is removed. Third, the gauze-bandage combination is not aesthetically pleasing nor designed for patient comfort in the application, retention, and removal of the gauze. Conventional means for adhering a chemically eluting product to the patient's tissue includes medicated adhesive patches and other similar products. However, these products also have their own disadvantages (e.g., prefilled patches cannot be customized by drug, dosage, or application area to an individual patient). These products also do not serve simultaneous purposes such as wound coverage or compression.

What is needed then are improvements to apparatuses and methods for applying pressure or substances to tissue in a controlled, customizable, and patient-centered manner.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is a customizable inflatable bolster apparatus. The apparatus may include a first leaf, a second leaf, and a balloon disposed between the first leaf and the second leaf. The balloon may include an inflation port and one or more tethers. The one or more tethers may be disposed within the balloon. In response to the balloon inflating, the one or more tethers may shape the balloon into a desired shape. The apparatus may apply a pressure to the tissue of patient and immobilize the superficial or deep tissue. The apparatus may be applied externally to a patient's skin or internally to a patient's mucosa or fascia. The apparatus may be resistant to bacteria.

Another aspect of the disclosure is a method of treating a patient with a customizable inflatable bolster apparatus. The method may include providing the customizable inflatable bolster apparatus. The apparatus may include a first leaf, a second leaf, and a balloon disposed between the first leaf and the second leaf. The balloon may include an inflation port and one or more tethers disposed within the balloon. In response to the balloon inflating, the one or more tethers may shape the balloon into a desired shape. The method may include disposing the first leaf against a tissue of the patient. The method may include adhering the first leaf to the tissue. The method may include inflating the balloon to a desired volume or pressure. Advantageously, the volume or pressure may be set after the balloon is affixed to the wound, allowing precise control over size, pressure, or wound contact.

Another aspect of the disclosure is another customizable inflatable bolster apparatus. The apparatus may include a first leaf, a second leaf, and a balloon disposed between the first leaf and the second leaf. The apparatus may apply a pressure to the tissue of patient and immobilize the tissue in response to the balloon inflating. The balloon may be instilled with a substance. The balloon may be semipermeable. The substance may elute into the tissue from the balloon. The balloon may cause a patient substance to be drawn into the balloon over time. The substance may include a moisturizing agent, growth factor, chemotherapeutic, or other chemical.

Another aspect of the disclosure includes a customizable inflatable bolster apparatus. The apparatus may include a baseplate and a balloon disposed on the baseplate. The balloon may include an inflation port. The baseplate may be customizable in shape and size. The balloon may herniate through an aperture in the baseplate.

Another aspect of the disclosure includes a customizable inflatable bolster apparatus. The apparatus may include an adherent material and a balloon disposed on the adherent material. As examples, the adherent material may be a wrap or undergarment. The adherent material may secure the balloon against tissue, such that inflating the balloon applies a pressure to the tissue of the patient and immobilizes the superficial or deep tissue.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1A is a perspective view illustrating one embodiment of a customizable inflatable bolster apparatus.

FIG. 1B is a cutaway side view illustrating one embodiment of a customizable inflatable bolster apparatus in a deflated state.

FIG. 1C is a top-down view illustrating one embodiment of a customizable inflatable bolster apparatus.

FIG. 2 is a cutaway side view illustrating one embodiment of a customizable inflatable bolster apparatus in an inflated state.

FIG. 3A is a top-down view illustrating one embodiment of a customizable inflatable bolster apparatus with a portion of the leaves removed.

FIG. 3B is a top-down view illustrating one embodiment of the customizable inflatable bolster apparatus of FIG. 3A with multiple sutures.

FIG. 3C is a top-down view illustrating one embodiment of the customizable inflatable bolster apparatus of FIG. 3B in an inflated state.

FIG. 4 is a top-down view illustrating one embodiment of a customizable inflatable bolster apparatus.

FIG. 5 is a close-up perspective view illustrating one embodiment of a leaf of a customizable inflatable bolster apparatus.

FIG. 6 is a top-down view illustrating one embodiment of a customizable inflatable bolster apparatus.

FIG. 7 is a cutaway side view illustrating one embodiment of a customizable inflatable bolster apparatus.

FIG. 8A is a top-down view illustrating one embodiment of a baseplate for a customizable inflatable bolster apparatus.

FIG. 8B is a top-down view illustrating another embodiment of a baseplate for a customizable inflatable bolster apparatus, wherein a custom shape has been created in the baseplate.

FIG. 8C is a top-down view illustrating an embodiment of a baseplate with a customer shape for a customizable inflatable bolster apparatus.

FIG. 8D is a top-down view illustrating an embodiment of a customizable inflatable bolster apparatus with a balloon included on the apparatus.

FIG. 8E is a top-down view illustrating another embodiment of a customizable inflatable bolster apparatus with a balloon included on the apparatus.

FIG. 9 is a cutaway side view illustrating one embodiment of a customizable inflatable bolster apparatus, wherein a balloon of the apparatus is herniating through a hole of the baseplate.

FIG. 10 is a cutaway side view illustrating an embodiment of a customizable inflatable bolster apparatus with a selectably detachable syringe adapter.

FIG. 11 is a top-down view illustrating an embodiment of a balloon incorporated into a lower body garment for a customizable inflatable bolster apparatus.

FIG. 12 is a top-down view illustrating an embodiment of a balloon incorporated into an upper body garment for a customizable inflatable bolster apparatus.

FIG. 13 is a top-down view illustrating an embodiment of a balloon apparatus incorporated into a wrap for a customizable inflatable bolster apparatus.

FIG. 14 is a side view illustrating an embodiment of an inflated customizable inflatable bolster apparatus positioned within a bodily cavity.

FIG. 15 is a side view illustrating an embodiment of a deflated customizable inflatable bolster apparatus positioned within a bodily cavity.

FIG. 15 is a top-down view illustrating an embodiment of a customizable inflatable bolster apparatus with a pressure indicator.

FIG. 16 is a top-down view illustrating an embodiment of a customizable inflatable bolster apparatus with a pressure indicator.

FIG. 17 is a top-down view illustrating an embodiment of a customizable inflatable bolster apparatus with a deflectable pressure indicator.

FIG. 18 is a top-down view illustrating an embodiment of a customizable inflatable bolster apparatus with a deflectable pressure indicator.

FIG. 19 is a top-down view illustrating an embodiment of a customizable inflatable bolster apparatus with a deflectable pressure indicator.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Reference throughout this specification to “one embodiment,” “an embodiment,” “another embodiment,” or similar language means that a particular feature, structure, or characteristic described in connection with the embodiment is included in at least one embodiment. Thus, appearances of the phrases “in one embodiment,” “in an embodiment,” “in some embodiments,” and similar language throughout this specification may, but do not necessarily, all refer to the same embodiment, but mean “one or more but not necessarily all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. As used herein, the term “a,” “an,” or “the” means “one or more” unless otherwise specified. The term “or” means “and/or” unless otherwise specified.

Multiple elements of the same or a similar type may be referred to as “Elements 102(1)-(n)” where n may include a number. Referring to one of the elements as “Element 102” refers to any single element of the Elements 102(1)-(n). Additionally, referring to different elements “First Elements 102(1)-(n)” and “Second Elements 104(1)-(n)” does not necessarily mean that there must be the same number of First Elements as Second Elements and is equivalent to “First Elements 102(1)-(n)” and “Second Elements (1)-(m)” where m is a number that may be the same or may be a different number than n.

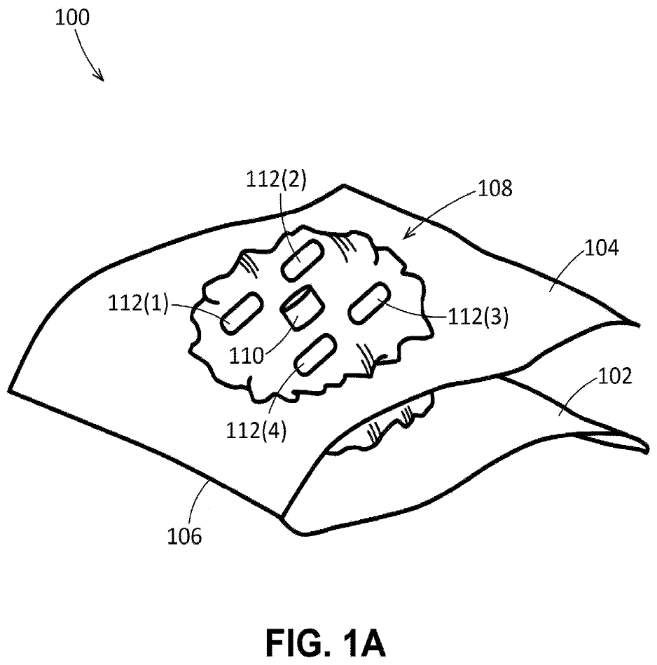

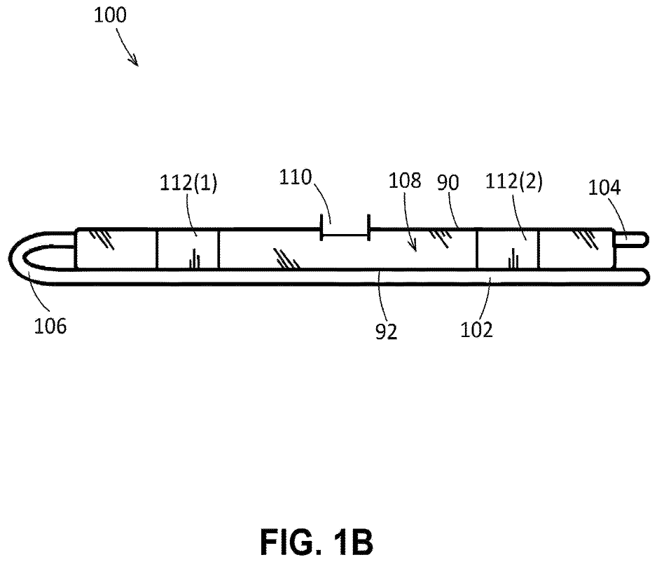

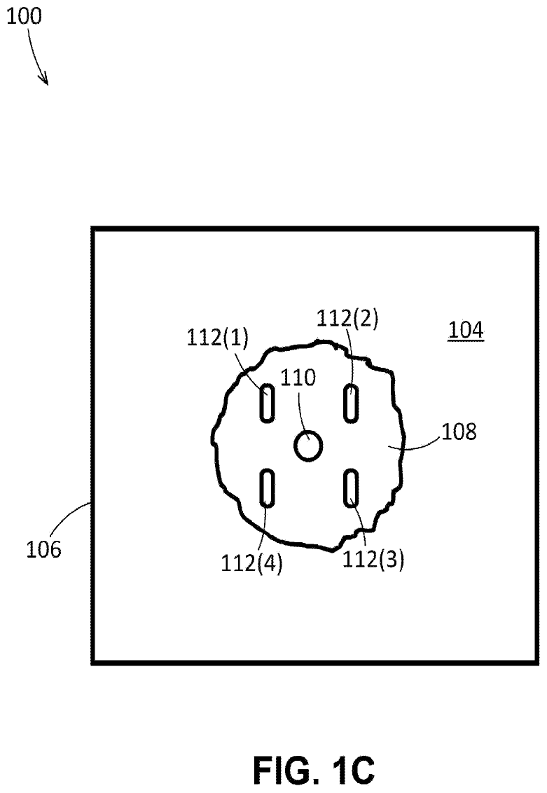

FIGS. 1A-C depict various views of one embodiment of a customizable inflatable bolster apparatus 100. The apparatus 100 may include a first leaf 102. The apparatus 100 may include a second leaf 104. The first leaf 102 and the second leaf 104 may join at a joint 106. The apparatus 100 may include a balloon 108. The balloon 108 may be disposed at least partially between the first leaf 102 and the second leaf 104. The balloon 108 may include an inflation port 110. The balloon 108 may include one or more tethers 112(1)-(n). For example, in FIG. 1A, the balloon 108 may include four tethers 112(1)-(4).

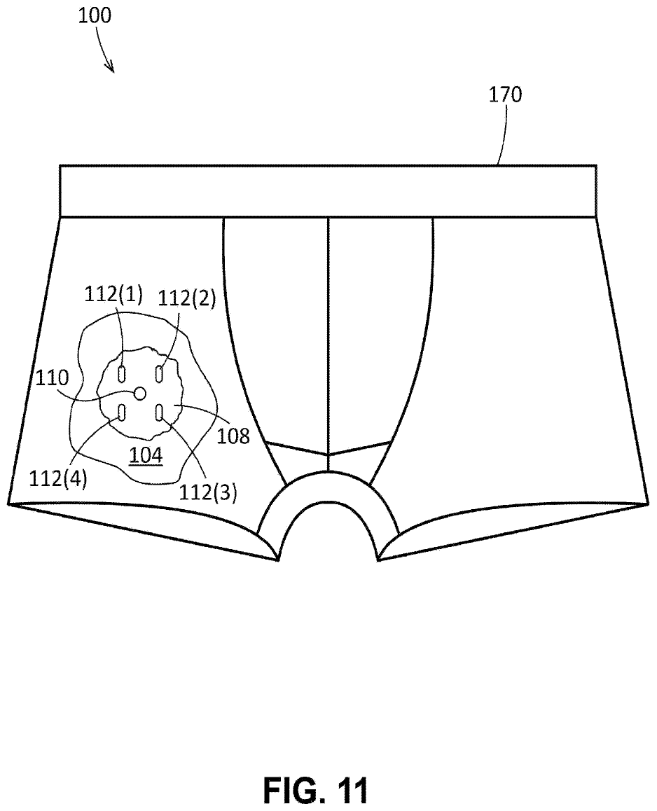

In one embodiment, the surface of the first leaf 102 may be disposed on a tissue of a patient. The first leaf 102 or the second leaf 104 may adhere to the tissue. The balloon 108 may be inflated, which may apply pressure to the tissue and may immobilize the tissue. The first leaf 102 or the second leaf 104 may adhere to the tissue via one or more sutures, an adhesive material (e.g., an acrylate), an adhesive ring, or some other adhesive. In other embodiments, and as discussed in greater detail below with reference to FIGS. 11-13, the balloon 108 is incorporated into or restrained by a wrap, strap, or garment. Advantageously, the volume or pressure may be set after the balloon is affixed to the patient or wound, allowing precise control over size, pressure, or wound contact.

Further details regarding the apparatus 100 are now disclosed. In one embodiment, the first leaf 102 or the second leaf 104 may include plastic, silicone, coated paper, woven fabric, latex, gauze, biologic or some other material. The plastic may include polyethylene, polyurethane, polyvinyl chloride (PVC), or some other plastic. The material of the first leaf 102 or the second leaf 104 may limit biofilm, bacteria, or debris. The material of the first leaf 102 or the second leaf 104 may include a layer that may limit friction or adhesion between the tissue and other components of the apparatus 100. As seen in FIGS. 1A-C, the first leaf 102 or the second leaf 104 may include a rectangular shape, but in other embodiments, the first leaf 102 or the second leaf 104 may include another shape (e.g., oval, circular, irregular shape, or another shape). The shape may be tailored to the specific shape of the tissue, wound, or other portion of the patient's body. The tailored shape may be created during the manufacturing process, or a medical worker may create the tailored shape (e.g., by cutting the first leaf 102 or second leaf 102 into the tailored shape). In some embodiments, the first leaf 102 and the second leaf 104 may be the same size or shape, and in other embodiments, the first leaf 102 and the second leaf 104 may be different sizes or shapes. In one embodiment, the first leaf 102 may be called the “inner leaf” and the second leaf 104 may be called the “outer leaf.” In one embodiment, the first leaf 102 or the second leaf 104 may be semipermeable. The first or the second leaf may be covered in an additional material for medical or aesthetic effect.

In some embodiments, the joint 106 may include the area of the apparatus 100 where the first leaf 102 and the second leaf 104 have been connected, joined, or otherwise disposed next to each other. In some embodiments, the first leaf 102 and the second leaf 104 may include different portions of the same piece, and the joint 106 may include a fold in the piece. In certain embodiments, the first leaf 102 and the second leaf 104 may include separate pieces, and the joint 106 may include the connection between the two pieces. The joint 106 may include a fold, welding, soldering, a seam, or some other connection. The joint 106 may act as a hinge. In some embodiments, the apparatus 100 may not include a joint 106 and the first leaf 102 and the second leaf 104 may be separate from each other.

In one embodiment, the balloon 108 may include a flexible bag that can be inflated with a fluid, such as a gas (air, oxygen, etc.) or a liquid. The balloon 108 may include a stretchable material such as a plastic, latex, rubber, biaxially oriented polyethylene terephthalate (BoPET), or some other material. The balloon 108 may inflate out to the edges of the first leaf 102 or second leaf 104 when volume is added. This inflation may be active (insertion of substance) or passive (draws in substance over time).

In some embodiments, the balloon 108 may include a substance. The balloon 108 may include a semipermeable material to allow elution of the substance. The substance may be disposed in the material of the balloon 108, inside the interior space created by the balloon 108, or in some other location, such as the material of the first or second lead 102, 104. The substance may include a pharmaceutical. For example, the pharmaceutical may include a pain medication (e.g., fentanyl), a moisturizing agent, a growth factor, or a chemotherapeutic. The substance may include a chemical. For example, the substance may apply heat or cold to the tissue via a chemical reaction. The balloon 108 may elute the substance into the patient's tissue. The balloon 108 may draw a substance from the patient's tissue into the balloon.

In some embodiments, the balloon 108 may include an inflation port 110. The inflation port 110 may include a tube into which a user may insert a fluid into the body of the balloon 108. The inflation port 110 may include a valve that may control the flow of the fluid. The valve may include a one-way valve that allows the fluid to enter the balloon 108 but not to exit (or, at least, not to exit without further manipulation).

In one or more embodiments, the balloon 108 may include one or more tethers 112(1)-(n). A tether 112 may include seal or a connector weld in the body of the balloon 108. The one or more tethers 112(1)-(n) may prevent the balloon 108 from inflating into a spherical shape, and may shape the inflated balloon 108 into a disc shape, puck shape, or some other desired shape. In other words, in response to the balloon 108 inflating, the one or more tethers 112(1)-(n) may shape the balloon 108 into a shape corresponding to an arrangement of the one or more tethers 112(1)-(n) within the ballon 108. The tethers 112(1)-(n) may allow the balloon 108 to provide substantially consistent pressure across a surface area of the apparatus 100 and against the tissue of the patient. A tether 112 may include a variety of shapes, such as a rectangle, a circle, an oval, a pill capsule, or an irregular shape. A tether 112 may curve, twist, or wrap. In some embodiments, a tether 112 may include a polymeric weld (e.g., a thermal weld, a radio-frequency (RF) weld, or a mechanical weld).

As shown in FIG. 1B, the balloon 108 may not penetrate through the first leaf 102, and the balloon 108 may penetrate through the second leaf 104. However, in some embodiments, the balloon 108 may penetrate through the first leaf 102, or the balloon 108 may not penetrate through the second leaf 104. As shown, the balloon 108 may have a first end 92 and a second end 90. In some embodiments, the first end 92 of the balloon 108 is secured to the first leaf 102 and the second end 90 of the balloon 108 projects at least partially through the second leaf 104.

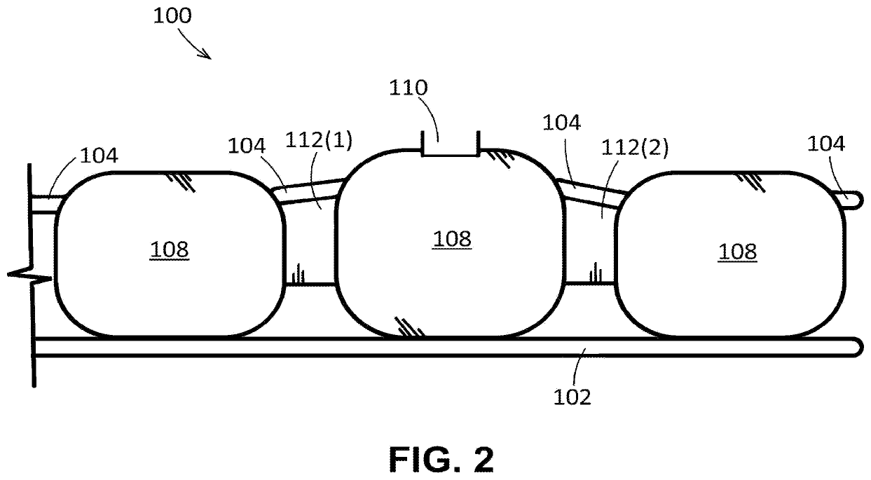

FIG. 2 depicts one embodiment of a apparatus 100 with the balloon 108 in an inflated state. As can be seen from FIG. 2, the balloon 108 may inflate and take on an expanded shape. However, the one or more tethers 112(1)-(n) may prevent the balloon 108 from taking on a spherical shape, and may, instead, take on a disc, puck, tube or other shape. As stated above, the disc, puck, or other desired shape may provide substantially consistent pressure across a surface area of the apparatus 100 and against the tissue of the patient.

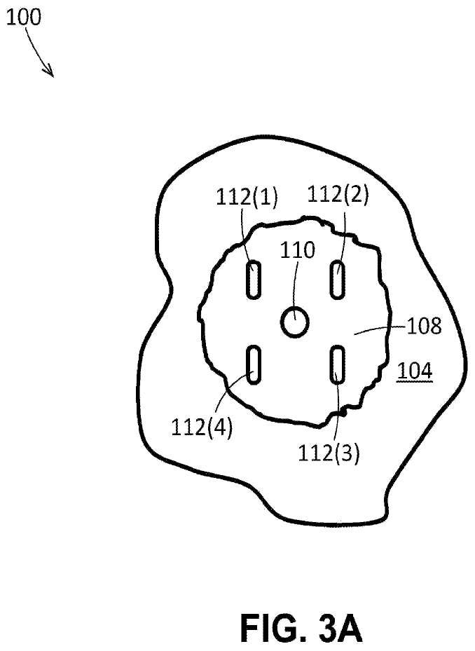

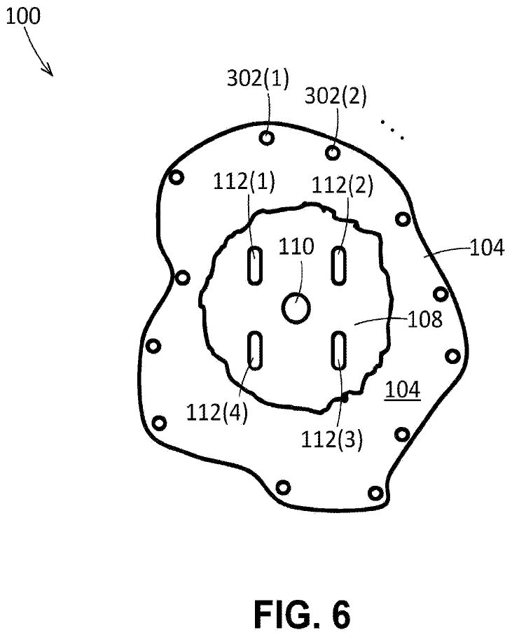

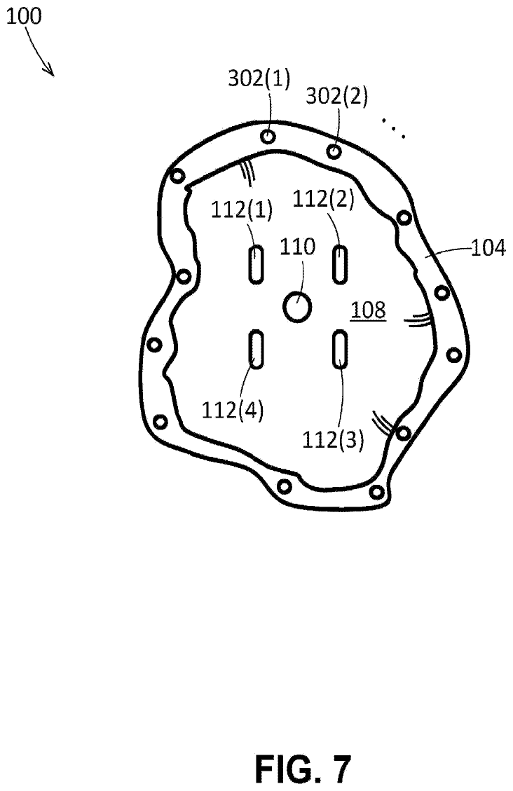

FIG. 3A depicts a apparatus 100 where a portion of the first leaf 102 and the second leaf 104 has been removed. The apparatus 100 may be customizable regarding shape. In one embodiment, customizing the shape of the apparatus 100 may include removing a portion of the first leaf 102 or the second leaf 104. For example, the first leaf 102 or the second leaf 104 may be trimmed to a specific shape or size to fit over the tissue of a patient. In response to the first leaf 102 and the second leaf 104 have been customized to the desired shape and size, a user may apply an adhesive to a portion of the apparatus. For example, as depicted in FIG. 3B, a user may apply one or more sutures 302(1)-(n) to hold the apparatus 100 in place. As seen in FIG. 3C, in response to the balloon 108 being inflated, the balloon 108 may expand to near the edges of the first leaf 102 or the second leaf 104.

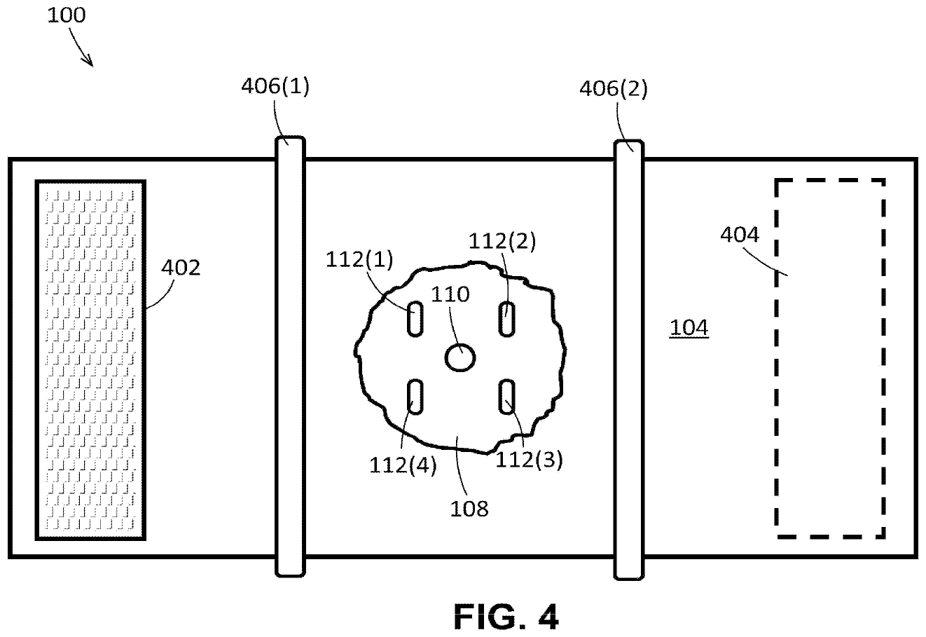

FIG. 4 depicts one embodiment of a apparatus 100. The apparatus 100 may include a first leaf 102 or a second leaf 104 that includes an elongated shape (e.g., as depicted in FIG. 4, an elongated rectangle). In one embodiment, the apparatus 100 may include a hook-and-loop fastener 402, 404. The hook portion 402 of the hook-and-loop fastener may be disposed on the opposite side of the first leaf 102 or the second leaf 104 from the loop portion 404 so that the apparatus can be wrapped around the patient's limb or some other surface of the patient. The hook portion 402 and the loop portion 404 may be disposed at opposite ends of the apparatus 100. In other embodiments, and as discussed in greater detail below with reference to FIGS. 11-13, the balloon 108 is incorporated into or restrained by a wrap, strap, or garment.

In one embodiment, the apparatus 100 may include one or more clips 406(1)-(n). A clip 406 may include an elongated clip that can clamp across at least a portion of the first leaf 102 and the second leaf 104 to hold the first leaf 102 and second leaf 104 together. The one or more clips 406(1)-(n) may help shape the balloon 108 when in an inflated state. A clip 406 may include a variety of shapes, including a clip 406 shaped like a hair clip or another shape. Different portions of the clip 406 may lock together to keep the clip 406 in a closed configuration.

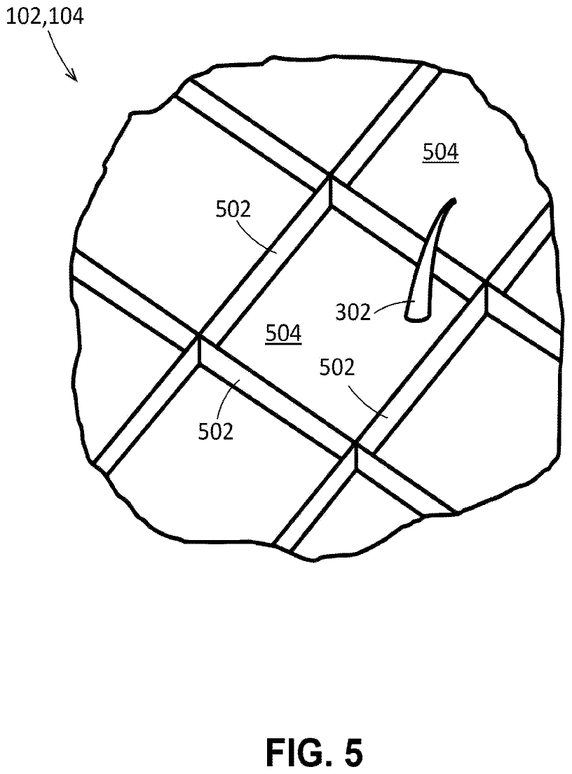

FIG. 5 depicts one embodiment of the first leaf 102 or the second leaf 104. The first leaf 102 or the second leaf 104 may include a grid of ridges 502 disposed on the surface 504 of the first leaf 102 or the second leaf 104. A ridge 502 may include an extra amount of material that may add extra strength to the first leaf 102 or the second leaf 104. The ridges 502 may prevent the first leaf 102 or the second leaf 104 from tearing. The ridges 502 may prevent a suture 302 from tearing further because the ridges 502 may contain the suture and prevent it from moving further over the surface 504 of the first leaf 102 or the second leaf 104. In one embodiment, a ridge 502 may include a silicone, metal (e.g., a metal wire), hardened plastic, or other material.

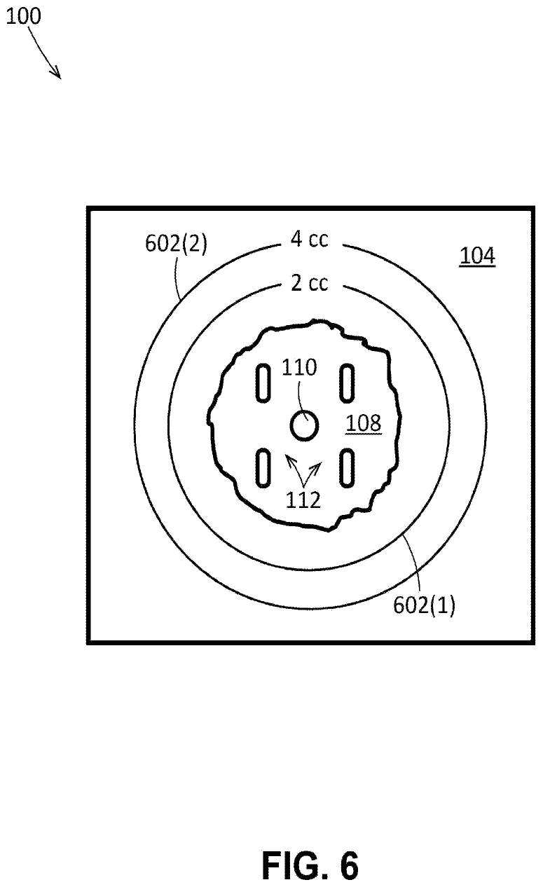

As depicted in FIG. 6, in one embodiment, the apparatus 100 may include one or more guides 602(1)-(n). A guide 602 may include a line disposed around the balloon 108. The guide 602 may include text indicating an inflation volume. In some embodiments, in response to the user inserting the indicated volume on a guide 602, the balloon 108 may inflate to the line of that guide 602. For example, as depicted in FIG. 6, in response to the user inserting 2 cubic centimeters (cc) of fluid, the balloon 108 may inflate to the first guide 602(1). In response to inserting 4 cc of fluid, the balloon 108 may inflate to the second guide 602(2).

In one embodiment, the volume or pressure of the balloon 108 may be given according to the table below:

| Inflation Volume (cc) | Balloon Volume (cm³) | Pressure (mmHg) |
|---|---|---|
| 1 | 1.7 | 3 |
| 2 | 2.04 | 9 |
| 3 | 2.176 | 13 |
| 4 | 2.301 | 19 |
| 5 | 2.4 | 25 |
| 6 | 2.5 | 30 |
| 7 | 2.54 | 34 |
| 8 | 2.6 | 37 |
| 9 | 2.63 | 39 |
| 10 | 2.7 | 40 |

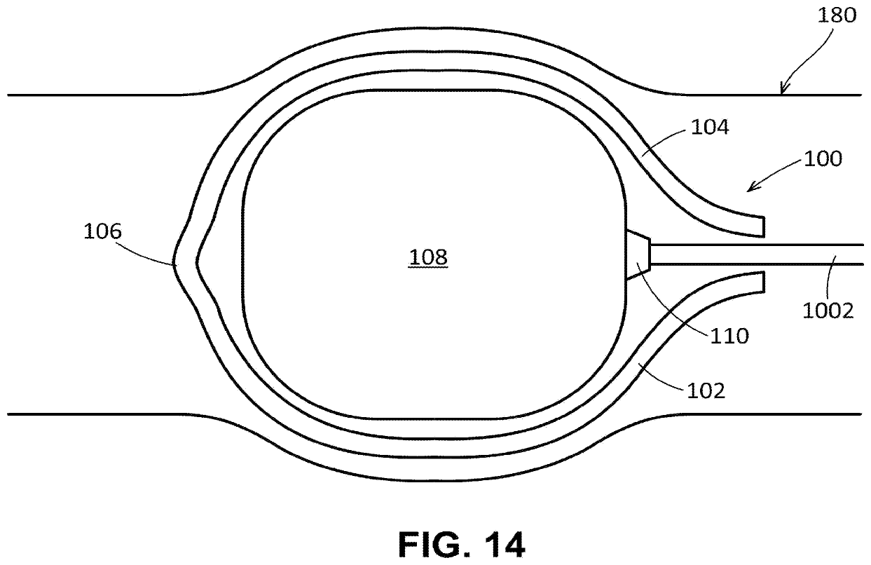

In some embodiments, the inflation volume or the balloon 108 volume or the balloon 108 pressure may include different numbers, different units, or other differences. For example, a balloon 108 volume for a corresponding inflation volume may include a higher number or a lower number. A balloon 108 pressure may include, for a corresponding inflation volume or balloon 108 volume, a higher or lower number. The balloon 108 pressure may include a pressure below a certain threshold, and the threshold may include a pressure that affects blood flow, skin perfusion pressure, or substance elution. In some embodiments, and as discussed in greater detail below with reference to FIGS. 14-19, the balloon pressure 108 may be indicated by a change to a particular portion of the balloon 108, such as a shape change or colorimetric change.

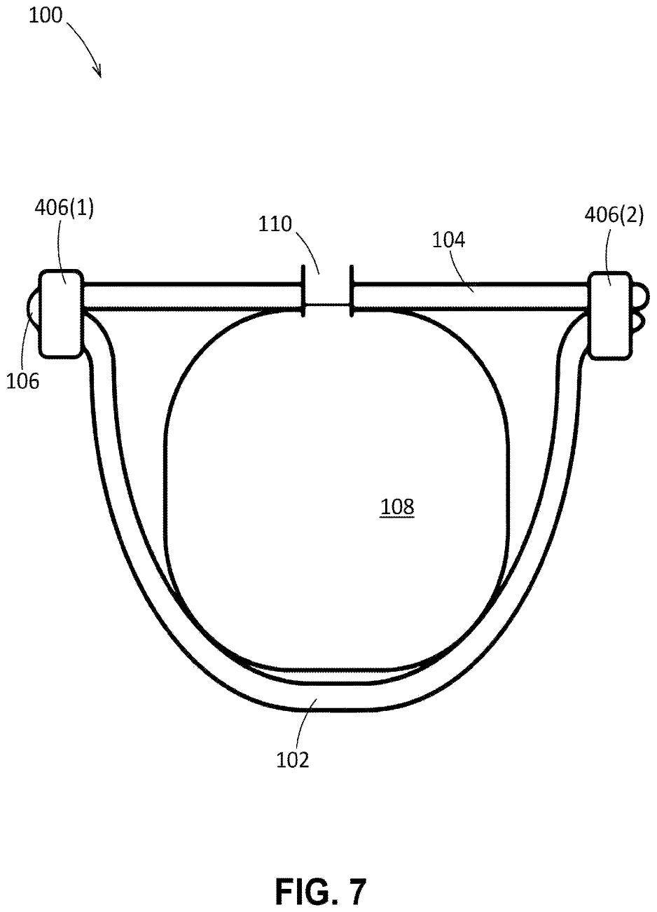

FIG. 7 depicts one embodiment of a apparatus 100. As can be seen from FIG. 7, the first leaf 102 and the second leaf 104 may bend differently in response to the inflation of the balloon 108. This difference in bending may be in response to the first leaf 102 having a different length than the second leaf 104. The difference may be in response to the first leaf 102 having a different amount of flexibility than the second leaf 104. The difference may allow the apparatus 100 to herniate (e.g., to intentionally fill a potential space or other application).

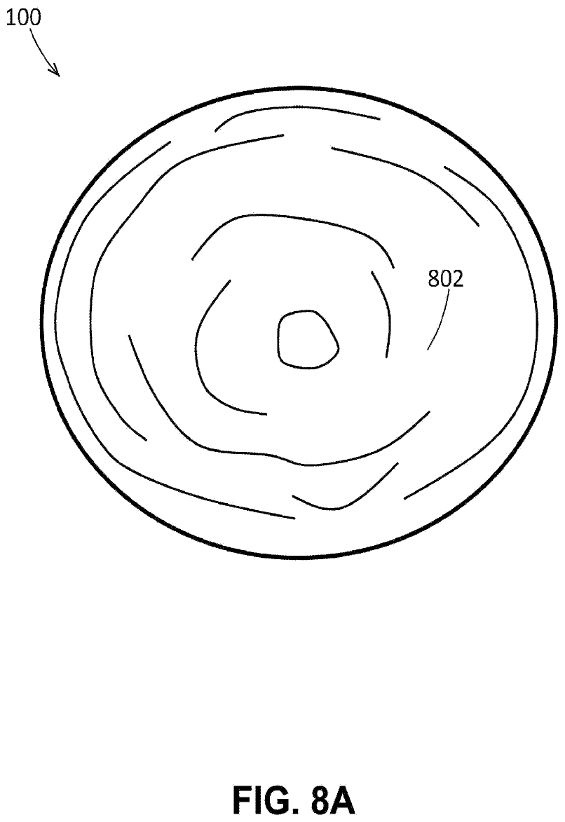

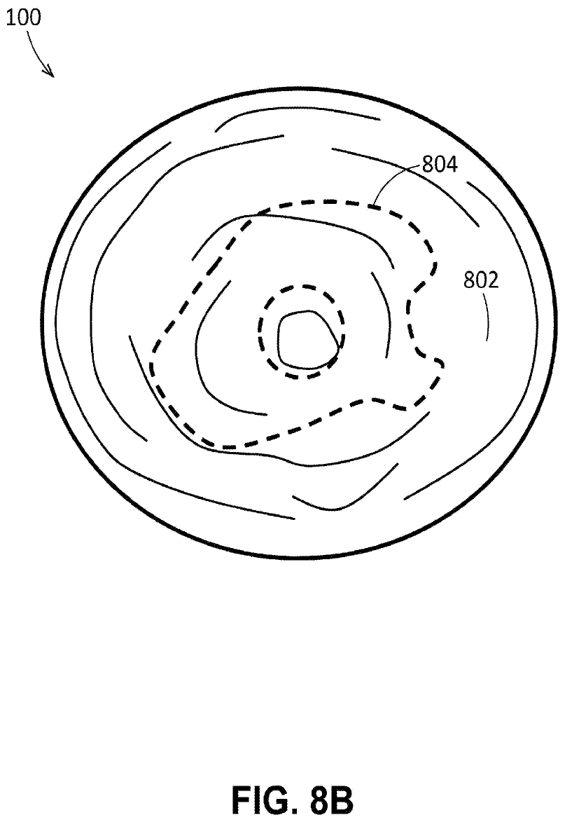

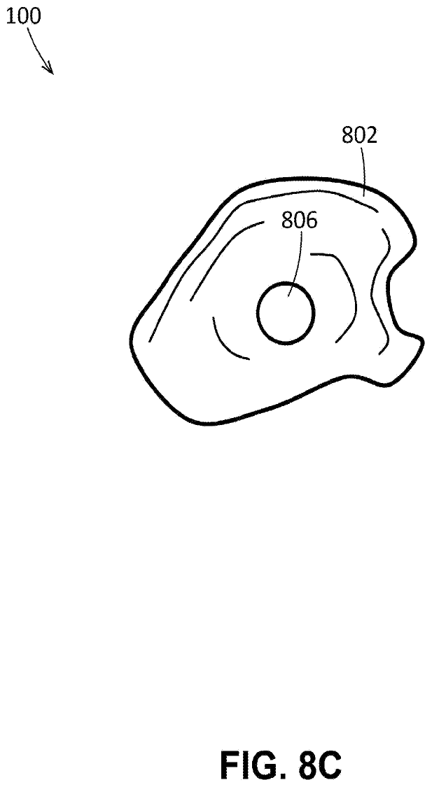

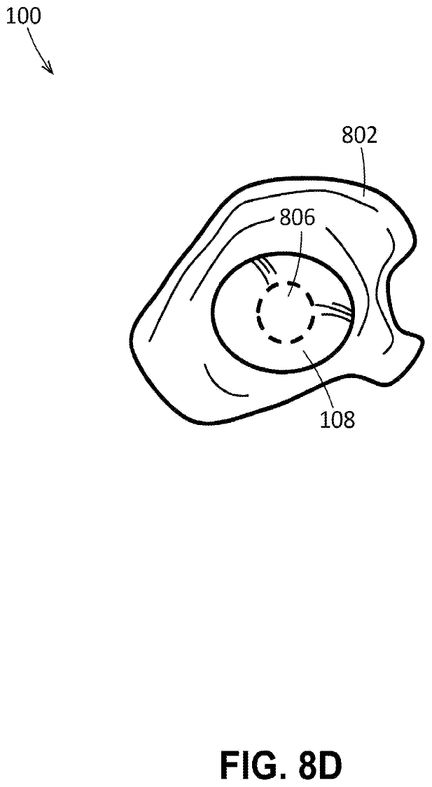

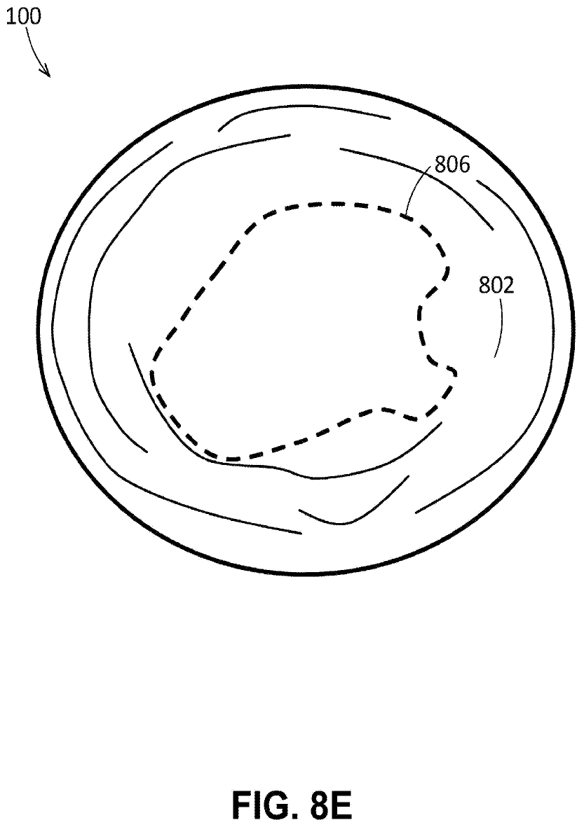

FIG. 8A depicts another embodiment of a apparatus 100. In some embodiments, the apparatus 100 may include a baseplate 802. The baseplate 802 may include a semi-rigid platform. The baseplate 802 may include a material that can be disposed over the skin of the patient and on which the balloon 108 may be disposed. The baseplate 802 may be similar to the first leaf 102, discussed above. The baseplate 802 may include a material that the first leaf 102 can include, as discussed above. The baseplate 802 may include a circular shape, as shown in FIG. 8A, or may include some other shape, such a square, rectangle, oval, or some other shape. In some embodiments, the baseplate 802 and the balloon 108 are provided as separate components, and are configured to be joined for application on a patient. For instance, the balloon 108 and the baseplate 802 may each include corresponding fastener components configured to facilitate selective attachment of the balloon 108 to the baseplate 802.

In some embodiments, the top side of the baseplate 802 (i.e., the side disposed away from the patient's skin) may be able to been sewn, adhered, or otherwise attachable to some other object. For example, the top side may include an adhesive, a hook-and-loop structure, or some other attachment structure. In one embodiment, the bottom side (i.e., the side disposed toward the patient's skin) may be coupleable to the patient's skin via an adhesive or some other attachment structure.

As shown in FIG. 8B, in some embodiments, a user may customize the shape or size of the baseplate 802. For example, the user may create a cutout from the baseplate 802 by cutting a shape 804 into the baseplate 802. FIG. 8C depicts one embodiment of the baseplate 802 having been cut into a shape. The user may also cut one or more apertures 806 into the baseplate 802. An aperture 806 may allow one or more objects to pass through the baseplate 802 and make contact with the patient's skin, a wound, or other area of the body of the patient.

As is shown in FIG. 8D, a balloon 108 may be disposed on the baseplate 802. The balloon 108 may be sewn to the baseplate 802. The balloon 108 may coupled to the baseplate 802 via a hook-and-loop structure. In some embodiments, the balloon 108 may be held in place by a second baseplate 802, a second leaf 104, a bandage, or some other component. In some embodiments, the balloon 108 may be integrated with the baseplate 802. In some embodiments, the balloon 108 may be disposed over the aperture 806. The aperture 806 may allow for the balloon 108 to herniate through the aperture 806 in response to being inflated.

As shown in FIG. 8E, the aperture 806 may be cut into the baseplate 802 in order to widen the hole through which the balloon 108 herniates. Advantageously, the area where the balloon 108 contacts the tissue may be precisely designated in such cases.

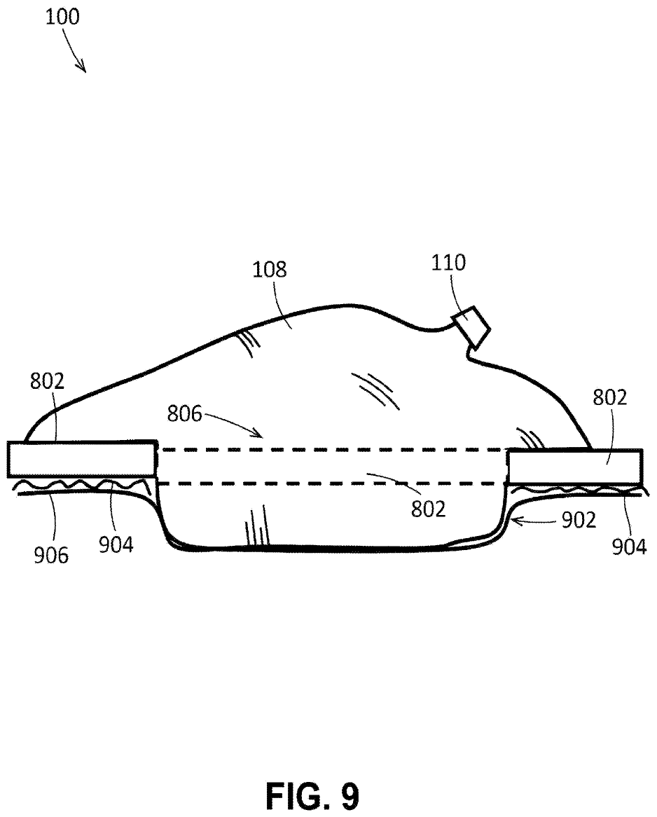

FIG. 9 depicts one embodiment of the balloon 108 disposed on the baseplate 802 having herniated into a patient's wound 902. A fluid can be introduced into the balloon 108 via in the inflation port 110 on the balloon 108. As can be seen in FIG. 9, in response to the balloon 108 inflating, a portion of the balloon 108 may swell and inflate upwards and another portion of the balloon 108 may swell and inflate downward through the aperture 806 and toward the patient's wound 902. The balloon 108, in its inflated state, may apply pressure to the wound 902. A substance in the balloon 108 may elute from the balloon 108 into the wound 902. As can also be seen in FIG. 9, in some embodiments, an adhesive 904 may be disposed between the patient's skin 906 and the baseplate 802 to selectively couple the baseplate 802 to the patient.

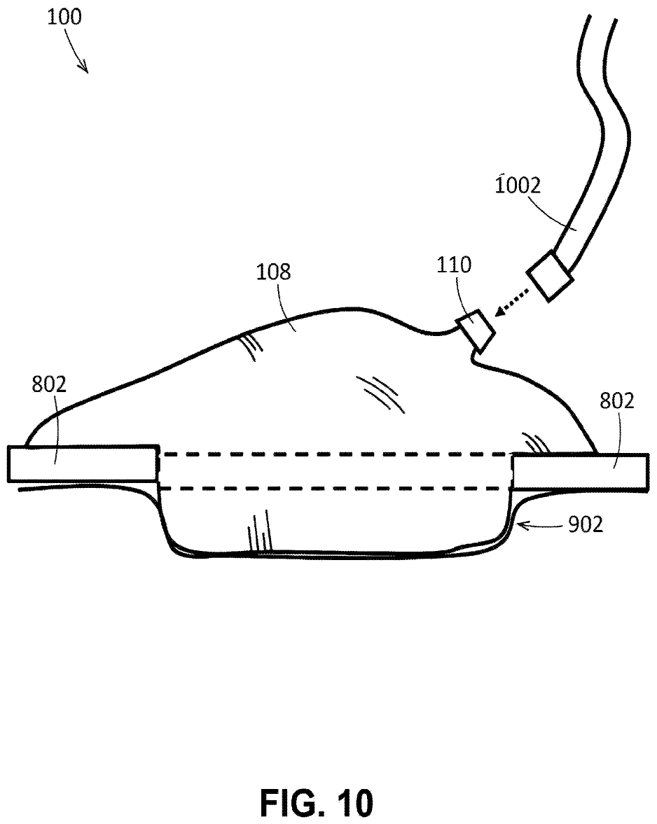

FIG. 10 depicts one embodiment of the apparatus 100 with the baseplate 802 and the balloon 108 disposed over a patient's wound 902. In one embodiment, a syringe adapter 1002 may be selectively disposable on the inflation port 110 of the balloon 108. The syringe adapter 1002 may include a flexible length of material that may include a channel through which fluid may pass. The syringe adapter 1002 may act as lengthener of the inflation port 110. The syringe adapter 1002 may allow a user to add fluid into the balloon 108 or remove fluid from the balloon 108 even if the balloon 108 is covered, for example, by a second leaf 104, a second baseplate 802, a bandage, or other materials. The syringe adapter 1002 may prevent disruption of the apparatus 100 in response to the balloon 108 being inflated. In some embodiments, depending on use (e.g., inflation using a fluid, pharmaceutical delivery, etc.), the syringe adapter 1002 may include different lengths.

In some embodiments, the customizable inflatable bolster apparatus 100 can be used in several different medical scenarios. For example, the apparatus 100 can be applied over a skin graft. The apparatus 100 can cover a wound to prevent bleeding. The apparatus 100 may cover a surgical incision. The apparatus 100 may be disposed internally on a patient's mucosa or fascia. The apparatus 100 can be disposed in the patient's mouth in connection with treating conditions of the gingivobuccal sulcus or other mouth conditions or injuries. The apparatus 100 may be disposed over the site where a tumor was removed from the patient. In further embodiments, the apparatus 100 may be disposed in a body cavity, such as a vagina or anus.

In some embodiments, the apparatus 100 may be selectively coupled to a patient's tissue. For example, the apparatus 100 may be sewn to the tissue (e.g., via one or more sutures), the apparatus 100 may be adhered to the tissue via an adhesive material (e.g., an acrylate), an adhesive ring, or some other adhesive. However, in other embodiments, non-adherence of the apparatus 100 to the patient's tissue may be desired. In such an embodiment, an interface may be disposed between the apparatus 100 and the patient's tissue. The interface may include a petroleum gauze (e.g., XEROFORM-brand petroleum gauze).

In one embodiment, multiple apparatuses 100 can be identically manufactured, and then the first leaves 102, the second leaves 104, or the baseplates 802 of the different apparatuses 100 can be trimmed or otherwise adjusted to the individual needs of different patients. In some embodiments, differently shaped apparatuses 100 may be manufactured for different purposes (e.g., for placement on convex tissue, for placement on concave tissue, for placement in a patient's mouth, for placement on mucosa or fascia, etc.). The differently shaped apparatuses 100 may have different alignments of the balloon 108 and inflation constraints. In one or more embodiments, different apparatuses 100 with differently shaped balloons 108, or differently sized, shaped, or positioned tethers 112(1)-(n) may be manufactured.

In some embodiments, the apparatus 100 may assist with skin perfusion pressure (SPP) or the ability to titrate pressure.

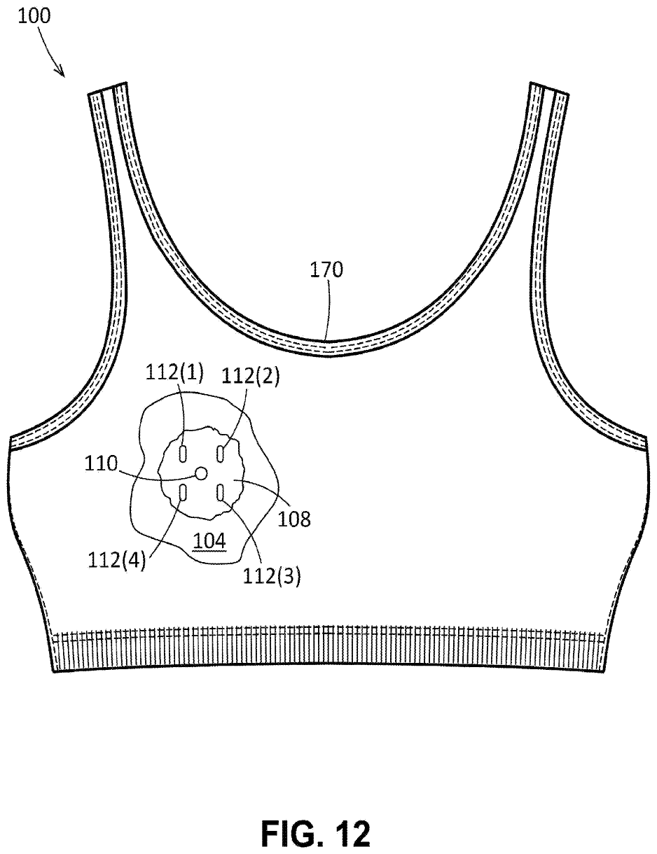

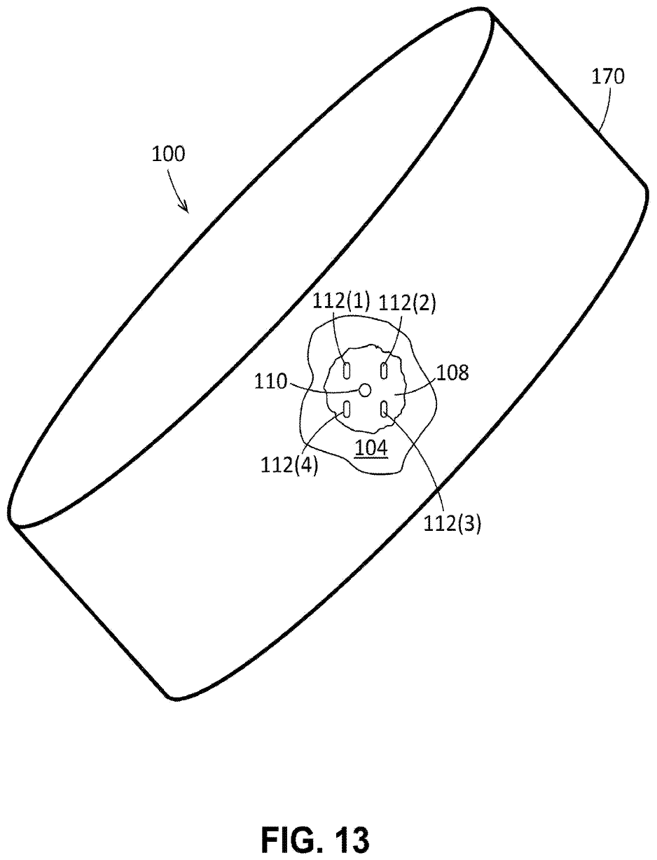

FIGS. 11-13 depict embodiments of the apparatus 100 with the balloon 108 incorporated into an (e.g., attached to, embedded within, restrained by, etc.) an adherent material 170. As a first example, and as depicted with reference to FIG. 11, the adherent material 170 may be a lower-body undergarment. In such cases, the balloon 108 may be incorporated into the adherent material 170 such that the balloon 108 provides pressure to an inguinal area after an endovascular procedure. As a second example, and as depicted with reference to FIG. 12, the adherent material 170 may be an upper-body undergarment, such as a bra. In such cases, the balloon 108 may be incorporated into the adherent material 170 such that the balloon 108 provides pressure to a mastectomy site. As a third example, and as depicted with reference to FIG. 13, the adherent material 170 may be a wrap or strap, such that the balloon 108 may provide pressure to a variety of anatomical sites.

In some cases, the balloon 108 is stitched within multiple layers of the adherent material 170. In other cases, the balloon 108 is attached to the adherent material 170 via a fastener such as Velcro. The balloon 108 may be incorporated into the adherent material 170 such that, when the adherent material 170 is secured to tissue, the balloon 108 is adhered to the tissue, and thus the balloon 108 may apply pressure to the tissue as described above. Depending on the implementation, where the balloon 108 is incorporated into the adherent material, the apparatus 100 still includes one or more of the other components discussed herein. For instance, the first and/or second leaves 102, 104 may still be included in order to support adhering the balloon 108 to the tissue. In other cases, the balloon 108 may be incorporated into the adherent material 170 such that the first and/or second leaves 102, 104 are not required.

In some embodiments, the apparatus 100 is applied to tissue via the adherent material 170, and then the pressure of the balloon 108 can be set (as discussed above) after application. Advantageously, this may provide for precise pressure control against the tissue.

Accordingly, present disclosure provides for an inflatable bolster apparatus 100 including the adherent material 170, which may be configured to be secured to payment tissue. The apparatus 100 may further include the balloon 108 disposed on the adherent material 170, the balloon 108 including the inflation port 110 and the one or more tethers 112(1)-(n). As discussed above, in response to the balloon 108 inflating, the one or more tethers 112(1)-(n) may shape the balloon 108 into a shape corresponding to an arrangement of the one or more tethers 112(1)-(n) within the balloon 108. In some cases, the adherent material 170 is a wrap. In other cases, the adherent material 170 is a lower body garment. In other cases still, the adherent material 170 is an upper-body garment.

The customizable inflatable bolster apparatus 100 disclosed herein may apply pressure to the tissue of a patient and immobilize the tissue while overcoming the disadvantages of the prior art. By using a balloon 108 that can be inflated to different volumes, the apparatus 100 allows control over the amount of pressure applied to the tissue of the patient. The pressure exerted by the balloon 108 can be adjusted over the course of treatment as needed, either by further inflating the balloon 108 or by releasing fluid from the balloon 108. This allows the user to adjust the treatment without having to remove the entire apparatus 100 and reapply it to the patient's tissue. The apparatus is resistant to the growth of bacteria and other undesired material, which prevents infection. The apparatus 100 is also more aesthetically pleasing than prior art solutions. Furthermore, the apparatus 100 may include various articles or designs for providing an aesthetically enhanced appearance.

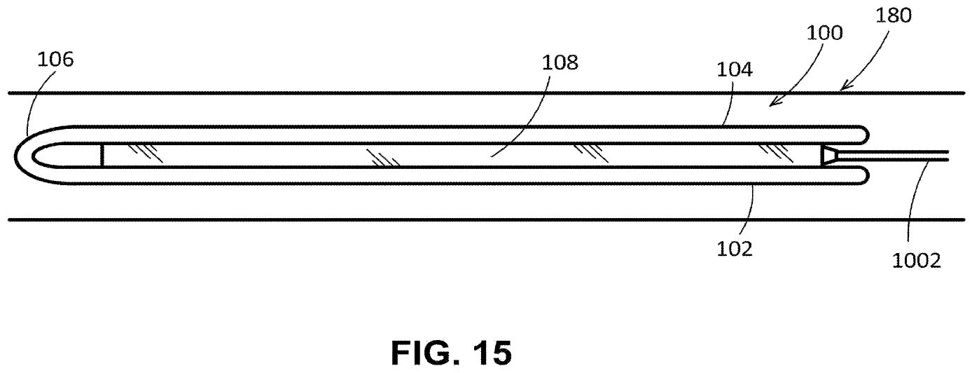

FIGS. 14-15 depict embodiments of the apparatus 100 as placed inside a bodily cavity 180. As a first example, the bodily cavity may be a blind anatomical pouch, such as an anus or vagina. As a second example, the bodily cavity 108 may be an anatomical tube, such as a gastro-intestinal tract or an airway. In some cases, the apparatus 100 is separated from the walls forming the anatomical cavity 108 by a graft. Advantageously, the first and second leaves 102, 104 may prevent the balloon 108 from adhering to the walls forming the bodily cavity 108 or the graft, depending on the implementation. As shown with reference to FIG. 15, the balloon 108 may be inserted or removed when in a deflated state. As shown with reference to FIG. 14, once located in a desirable position within the bodily cavity 108, the balloon 108 may be inflated in order to provide a desirable amount of pressure to the walls forming the bodily cavity 108.

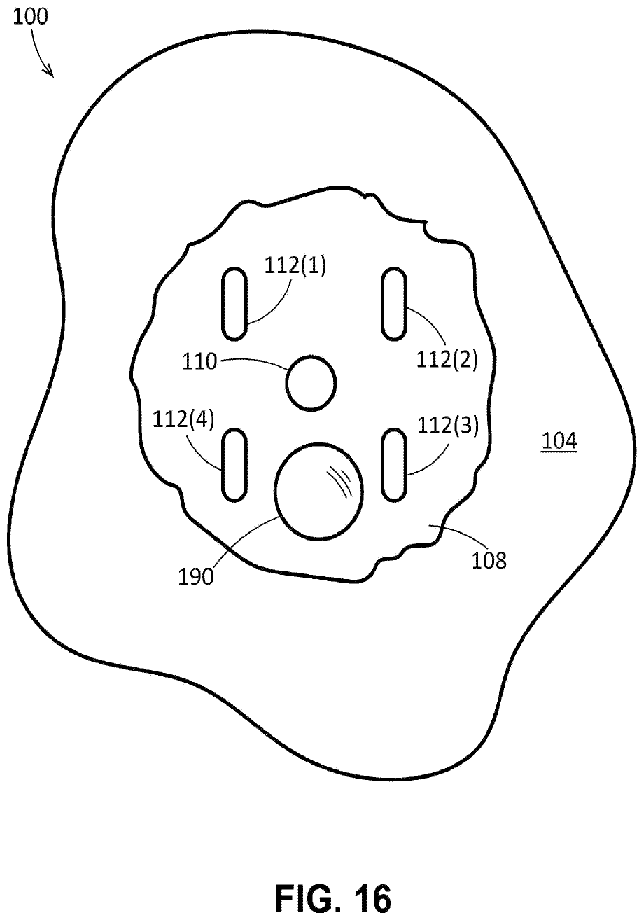

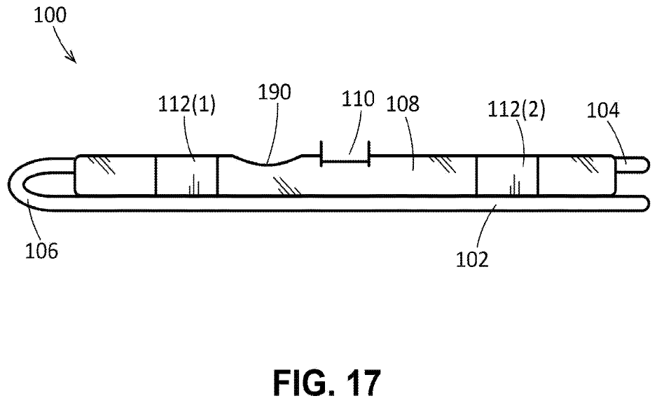

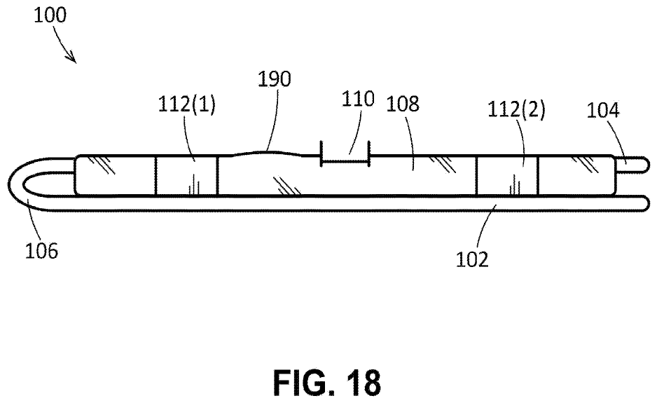

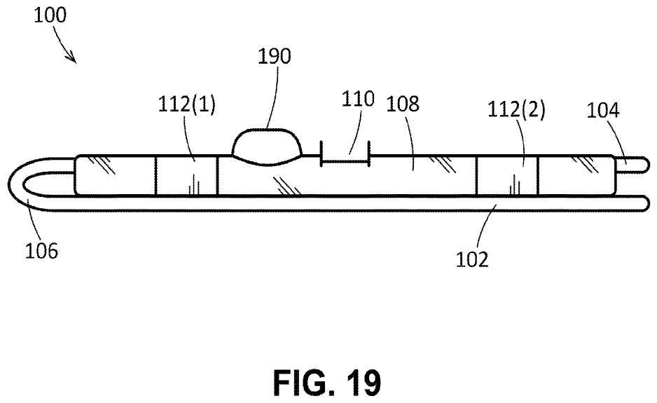

FIGS. 16-19 depict embodiments of the apparatus 100 including a pressure indicator 190. In some embodiments, the pressure indicator is disposed on the balloon 108. In some cases, the pressure indicator 190 includes a pressure sensor, and the pressure indicator 190 is configured to display a pressure measurement or display a light with a color that corresponds to various pressure measurements (e.g., a colorimetric sensor). In other cases, the pressure indicator 190 is made of a chromogenic material that changes color when under various pressures (particularly in cases where the pressure indicator 190 is incorporated into the material of the balloon 108). In other cases still, the pressure indicator 190 is incorporated into the material of the balloon 108 and made of a material separate from the balloon 108 that deforms depending on the pressure within the balloon 108. As a first example, and with reference to FIG. 17, the pressure indicator 190 may have a default configuration when the balloon is at a low pressure. As a second example, and with reference to FIG. 18, as pressure within the balloon 108 is increased, the pressure indicator 190 may begin deforming outward, thus indicating the increasing pressure within the balloon 108. As a third example, and with reference to FIG. 19, the pressure indicator 190 may reach a particular deformation shape when the balloon 108 reaches a desired reference pressure, a maximum pressure, and so on.

In further embodiments, the indicator 190 may include an apparatus that pops out (e.g., rapidly advance from) the balloon 108 when a certain pressure is achieved. In further embodiments still, a manometer may be applied at the inflation port 110 or syringe.

Furthermore, the customizable inflatable bolster apparatus 100 may deliver a substance to a patient's tissue. For example, the apparatus 100 may elute a pharmaceutical, moisture, growth factors, or chemotherapeutic into the patient's tissue.

As generally discussed herein, the balloon 108 may be referred to as being actively inflated, by way of air being deposited within the balloon 108. However, in further embodiments, the balloon 108 self-expands (e.g., passively inflates) rather than actively inflates. For instance, the balloon 108 may be made of a memory foam. In further embodiments, the balloon 108 is made of a material with phase-change properties, such that the balloon 108 hardens or softens depending on the temperature of the balloon 108. Advantageously, the balloon 108 may be made of a material with phase-change properties such that the balloon 108 is harder at colder temperatures, making the apparatus 100 more easily manipulated, while the balloon 108 becomes softer at a body temperature, making the pressure that the apparatus 100 applies to the tissue less intrusive on, or better conforming to, the user.

Thus, although there have been described particular embodiments of the present invention of a new and useful CUSTOMIZABLE INFLATABLE BOLSTER APPARATUS, it is not intended that such references be construed as limitations upon the scope of this invention.

What is claimed is:

1. An inflatable bolster apparatus, comprising:
a first leaf;
a second leaf; and
a balloon disposed between the first leaf and the second leaf, the balloon including:
an inflation port, and
one or more tethers disposed within the balloon,
wherein in response to the balloon inflating, the one or more tethers shape the balloon into a shape corresponding to an arrangement of the one or more tethers within the balloon.

2. The apparatus of claim 1, wherein:
the balloon includes a substance; and
the substance includes at least one of:
a pharmaceutical,
a moisturizing agent,
a growth factor, and
chemotherapeutic.

3. The apparatus of claim 1, wherein at least one of the first leaf or the second leaf includes at least one of:
a semipermeable membrane;
a grid of edges; and
a friction-limiting material that limits friction between the apparatus and a patient's tissue.

4. The apparatus of claim 1, wherein the first leaf and the second leaf includes a microorganism-resistant material.

5. The apparatus of claim 1, further comprising a clip disposed around at least a portion of the first leaf and the second leaf.

6. The apparatus of claim 1, further comprising guide indicia disposed on the second leaf.

7. A method of treating a patient with a customizable inflatable bolster apparatus, comprising:
providing the customizable inflatable bolster apparatus, including:
a first leaf,
a second leaf, and
a balloon disposed between the first leaf and the second leaf, the balloon including an inflation port and one or more tethers disposed within the balloon, wherein in response to the balloon inflating, the one or more tethers shape the balloon into a shape corresponding to an arrangement of the one or more tethers within the balloon;
disposing the first leaf against a tissue of the patient;
adhering the first leaf to the tissue; and
inflating the balloon to a desired volume.

8. The method of claim 7:
wherein the balloon further includes a substance; and
further including eluting the substance from the balloon into the tissue of the patient.

9. The method of claim 7, wherein the tissue comprises a skin graft.

10. The method of claim 7, wherein the tissue comprises skin or a mucosa of the patient.

11. The method of claim 7, wherein the tissue comprises a potential space.

12. The method of claim 7, further comprising customizing the shape of the apparatus by removing at least a portion of the first leaf and the second leaf.

13. A customizable inflatable bolster apparatus, comprising:
a baseplate; and
a balloon disposed on the baseplate, the balloon including an inflation port.

14. The apparatus of claim 13, wherein:
the baseplate includes an aperture; and
the balloon is configured to herniate through the aperture.

15. The apparatus of claim 13, further including a syringe adapter that selectively couples to the inflation port.

16. The apparatus of claim 13, wherein the balloon is selectively coupled to the baseplate.

17. An inflatable bolster apparatus, comprising:
an adherent material configured to be secured to patient tissue; and
a balloon disposed on the adherent material, the balloon including:
an inflation port; and
one or more tethers disposed within the balloon,
wherein in response to the balloon inflating, the one or more tethers shape the balloon into a shape corresponding to an arrangement of the one or more tethers within the balloon.

18. The apparatus of claim 17, wherein the adherent material is a wrap.

19. The apparatus of claim 17, wherein the adherent material is a lower-body garment.

20. The apparatus of claim 17, wherein the adherent material is an upper-body garment.
